(19) United States
(12) Patent Application Publication
(10) Pub. No.: US 2022/0289824 A1
(43) Pub. Date: Sep. 15, 2022

(54) TGF-beta TRAP

(71) Applicant: NANTBIO, INC., Culver City, CA (US)

(72) Inventors: Philip LIU, Culver City, CA (US); Wendy HIGASHIDE, Culver City, CA (US); C. Anders OLSON, Culver City, CA (US); Kayvan NIAZI, Culver City, CA (US)

(21) Appl. No.: 17/635,625

(22) PCT Filed: Aug. 14, 2020
(86) PCT No.: PCT/US20/46311

§ 371 Date: Feb. 15, 2022

Related U.S. Application Data
(60) Provisional application No. 62/887,272, filed on Aug. 15, 2019.

(51) Int. Cl. C07K 14/71; C07K 16/22; C12N 5/0783; A61K 9/00

(52) U.S. Cl. CPC C07K 14/71; C07K 16/22; C12N 5/0646; A61K 9/0019; C07K 2317/53; C07K 2317/76; C07K 2319/30; C12N 2510/00

(57) ABSTRACT

Compositions and methods are provided for inhibiting TGF-β. Trap molecules are provided in which a ligand-binding domain of transforming growth factor-beta receptor type 2 (TGFβRII) is fused to an immunoglobulin Fc domain that contains an N-terminal immunoglobulin hinge region where at least one unpaired cysteine residue of said hinge region is replaced by a serine residue.

### CROSS REFERENCE

This application claims the benefit of U.S. Provisional Patent Application No. 62/887,272, filed Aug. 15, 2019, the entire contents of which are incorporated by reference herein.

### REFERENCE TO SEQUENCE LISTING

This application contains a Sequence Listing submitted as an electronic text file named “8774-14-PCT_Seq_Listing_ST25.txt”, having a size in bytes of 68 bytes, and created on Aug. 14, 2020. The information contained in this electronic file is hereby incorporated by reference in its entirety pursuant to 37 CFR § 1.52(e)(5).

### FIELD

Compositions and methods are provided for inhibiting the activity of transforming growth factor beta (TGF-β).

### BACKGROUND

TGF-βs are a family of multifunctional cytokines involved in cell proliferation and differentiation, embryonic development, extracellular matrix formation, bone development, wound healing, hematopoiesis, and immune and inflammatory responses. TGF-β expression has been studied in a variety of cancers, including prostate, breast, lung, colorectal, pancreatic, liver, skin cancers, and gliomas. In early-stage tumors, increased levels of TGF-β correlate with a favorable prognosis, whereas in advanced tumors they correlate with tumor aggressiveness and poor prognosis. More specifically, TGF-β promotes cancer cell motility, invasion, epithelial-to-mesenchymal transition (EMT), and cell stemness, favoring tumor progression and metastasis.

There are three isoforms of human TGF-β: TGF-β1, TGF-β2 and TGF-β3. Each isoform occurs as a 25 kDa homodimer where two 112 amino acid monomers are joined by an inter-chain disulfide bridge. TGF-β1 differs from TGF-β2 and TGF-β3 by 27 and 22 (mainly conservative) amino acid changes respectively. Deregulation of TGF-βs is implicated in numerous conditions, such as birth defects, cancer, chronic inflammatory, autoimmune and fibrotic diseases.

TGF-β signaling occurs through a superfamily of receptors that can be divided into two groups of transmembrane proteins: the type I and the type II serine/threonine kinases. Whether the type I or the type II receptor binds first is ligand-dependent, and the second type I or type II receptor is then recruited to form a heteromeric signaling complex. A functional receptor complex has one dimeric ligand interacting with two type I and two type II receptors. Type I receptors are referred to as the Activin-like Kinases (ALKs), while the type II receptors are named for the ligands they bind. The Type II receptor binds TGF-β1 and TGF-β3 with high affinity but binds TGF-β2 with much lower affinity. The Type I and Type II receptors together form a heterodimeric signaling complex that is essential for the transduction of the anti-proliferative signals of TGF-β. A TGF-β type III receptor also exists but its cytoplasmic domain lacks an obvious signaling motif and the receptor may not be involved directly in signal transduction.

Systemic inhibition of TGF-β has been achieved using a variety of approaches, including antibodies and so-called trap molecules that contain TGF-β receptor domains fused to antibody Fc domains. See, for example, Gramont et al., Oncoimmunology 6:e1257453 (2017) and De Crescenzo et al., “Engineering TGF-β Traps: Artificially Dimerized Receptor Ectodomains as High-affinity Blockers of TGF-β Action” in Transforming Growth Factor-β in Cancer Therapy, Volume II pp 671-684 (2008). Studies with knockout mice have demonstrated that systemic loss of TGF-β function can lead to decreased wound healing, loss of immune regulation and an increased inflammatory response. Accordingly, it would be preferable to inhibit TGF-β activity in a tumor-localized manner.

### SUMMARY

What is provided is a TGF-β trap, containing an immunoglobulin Fc domain fused to a ligand-binding domain of transforming growth factor-beta receptor type 2 (TGFβRII), where the trap does not contain a Sushi domain, and where the immunoglobulin Fc domain further contains an N-terminal immunoglobulin hinge region in which at least one unpaired cysteine residue of the hinge region is replaced by a serine residue. In one aspect, the TGFβRII is linked to the Fc region via a flexible peptide linker moiety. In certain non-limiting embodiments, the ligand-binding domain of TGFβRII is linked to the carboxy-terminus (C-terminus) of the Fc domain via the flexible peptide linker moiety, while in other non-limiting embodiments the ligand-binding domain is linked to the amino-terminus (N-terminus) of the hinge region of the Fc domain via the flexible peptide linker moiety. In some aspects, the C27 residue of the hinge region is replaced by a serine residue.

In certain embodiments, the trap may have the structure: NH₂-hinge-Fc-linker-TGFβRII-CO₂H. In certain embodiments, the trap may have the structure NH₂-TGFβRII-linker-hinge-Fc-CO₂H. In one aspect, the flexible linker comprises G4S repeats, such as three, four, five, or more G4S repeats.

In one embodiment, the trap comprises an amino acid sequence at least 85% identical to SEQ ID NO:24 or at least 85% identical to SEQ ID NO:25.

Also provided are nucleic acid molecules encoding a trap as described above, where the trap may optionally be fused to an N-terminal peptide signal sequence. The nucleic acid molecules can be contained within an expression vector. The promoter in the expression vector can be operably linked to the nucleic acid molecule encoding the trap. In certain embodiments, the promoter can be an inducible promoter. In one aspect the inducible promoter is a TGF-β-inducible promoter. In certain aspects, the promoter can be constitutively active, such as a cytomegalovirus (CMV) promoter.

Also provided are host cells transformed with a vector as described above. In certain, non-limiting embodiments, the host cell can be a mammalian cell, such as a CHO cell or a human cell such as an NK-92 cell.

In other embodiments, the invention is related to, methods for inhibiting the activity of TGF-β in a subject or patient, comprising administering an effective amount of a trap as described above to a subject in need thereof.

In further embodiments, the invention is related to methods for inhibiting the activity of TGF-β in a subject, comprising administering to a subject in need thereof a composition containing host cells as described above, in an amount sufficient to produce an effective amount of the TGF-β trap.

In other embodiments, the invention is related to methods for treating a neoplasia in a subject, comprising administering to a subject in need thereof a therapeutically effective amount of a composition containing host cells as described above. In one aspect, the host cells can be administered parenterally, intravenously, peritumorally, or by infusion.

In any of the methods of treatment disclosed herein, an additional therapeutic agent can be administered to the subject or patient.

### BRIEF DESCRIPTION OF THE DRAWINGS

FIGS. 1A and 1B show inhibition of TGF-β response by constitutively active TGF-β Trap constructs. The CMV-driven expression constructs contained the Sushi domain (Sushi), unmodified hinge (AltH), Fc domain (Fc) with or without the TGFBRII (Trap) vs. the modified hinge ((C27S)H), Fc with or without the TGFBRII (Trap).

FIGS. 2A and 2B show inhibition of TGF-β response by TGF-β inducible Trap constructs. 293T cell lines stably expressing TGF-β induced luciferase were transfected with TGF-β response element (TGFBRE)-driven expression constructs comparing the Sushi domain (Sushi), unmodified hinge (AltH), Fc domain (Fc) with or without the TGFBRII (Trap) vs. the modified hinge ((C27S)H), Fc with or without the TGFBRII (Trap).

FIG. 3 shows testing of TGF-β trap expression constructs in stably transfected 293T cells. 293T cell lines stably expressing TGF-β induced luciferase were transfected with TGF-β response element (TGFBRE)-driven expression constructs with the Sushi domain (Sushi), the unmodified hinge (AltH), Fc with or without the TGFBRII (Trap) vs. the modified hinge ((C27S)H), Fc with or without the TGFBRII (Trap).

FIG. 4 show testing of N-terminal and C-terminal fusions with the TGF-β trap in stably transfected 293T cells. 293T cell lines stably expressing TGF-β induced luciferase were transfected with CMV-driven expression constructs comparing the C-terminal (C-term) vs. N-terminal (N-term) TGF-βRII (Trap) Fc-fusion proteins, with the modified hinge ((C27S)H).

FIGS. 5A and 5B show the ability of the TGF-β trap to neutralize TGF-β2. 293T cell lines stably expressing TGF-β induced luciferase were transfected with CMV-driven or TGF-β response element (TGFBRE)-driven expression constructs then stimulated with a titration of TGF-β2.

FIGS. 6A and 6B show the ability of the TGF-β trap to neutralize mouse TGF-β2. 293T cell lines stably expressing TGF-β induced luciferase were transfected with CMV-driven or TGF-β response element (TGFBRE)-driven expression constructs (DNA sequences shown in SEQ ID NOs: 8 and 16) then stimulated with a titration of mouse TGF-β1 (mTGF-β1). Cells were incubated overnight, and the resulting luciferase activity was measured after 24 hours.

FIG. 7 shows the effects of Sushi domain and hinge modification on production of TGF-β-trap. 293T cell lines were transfected with CMV-driven TGFBRII trap Fc fusion protein expression constructs containing the Sushi domain and original unmodified hinge sequence (AltH), no Sushi domain with the AltH, or no Sushi with the modified hinge ((C27S)H). The cells were rested and cultured in serum free medium for 24 hours, and the resulting supernatants were concentrated and the levels of human IgG FC were measured.

### DETAILED DESCRIPTION

TGF-β trap molecules are provided that contain an immunoglobulin Fc domain linked to a ligand-binding domain of transforming growth factor-beta receptor type 2 (TGFβRII). The immunoglobulin Fc domain contains an improved N-terminal immunoglobulin hinge region where at least one unpaired cysteine residue of the hinge region is replaced, for example by a serine residue. In certain embodiments, the trap molecules do not contain a Sushi domain. Nucleic acid molecules encoding the trap molecules are provided—together with vectors and expression constructs—that can be used to transfect cells and express the trap molecules. Also provided are cells transfected with nucleic acid constructs where expression of the trap molecules are under the control of a TGF-β-inducible promoter. Methods of using these traps, nucleic acid molecules and transfected cells for treating disease, such as cancer, also are provided.

### Ligand Binding Domain

The trap molecules contain a TGF-β binding domain derived from the extracellular ligand binding portion of the transforming growth factor beta receptor II (TGFβRII). The amino acid sequence of TGFβRII is:

(SEQ ID NO: 1)
MGRGLLRGLWPLHIVLWTRIASTIPPHVQKSVNND
MIVTDNNGAVKFPQLCKFCDVRFSTCDNQKSCMSN
CSITSICEKPQEVCVAVWRKNDENITLETVCHDPK
LPYHDFILEDAASPKCIMKEKKKPGETFFMCSCSS
DECNDNIIFSEEYNTSNPDLLLVIFQVTGISLLPP
LGVAISVIIIFYCYRVNRQQKLSSTWETGKTRKLM
EFSEHCAIILEDDRSDISSTCANNINHNTELLPIE
LDTLVGKGRFAEVYKAKLKQNTSEQFETVAVKIFP
YEEYASWKTEKDIFSDINLKHENILQFLTAEERKT
ELGKQYWLITAFHAKGNLQEYLTRHVISWEDLRKL
GSSLARGIAHLHSDHTPCGRPKMPIVHRDLKSSNI
LVKNDLTCCLCDFGLLRLDPTLSVDDLANSGQVGT
ARYMAPEVLESRMNLENVESFKQTDVYSMALVLWE
MTSRCNAVGEVKDYEPPFGSKVREHPCVESMKDNV
LRDRGRPEIPSFWLNHQGIQMVCETLTECWDHDPE
ARLTAQCVAERFSELEHLDRLSGRSCSEEKIPEDG
SLNTTK

where the extracellular domain of the receptor is underlined. The trap molecules may contain some or all of the extracellular domain, provided that the domain retains the ability to binding the ligand. Advantageously, the trap molecule contains the ligand binding sequence shown below. The skilled artisan will recognize that amino acids may be added or deleted from the N- and/or C-termini of the domain, provided that the ligand binding property of the domain is retained.

(SEQ ID NO: 2)
IPPHVQKSVNNDMIVTDNNGAVKFPQLCKFCDVRF
STCDNQKSCMSNCSITSICEKPQEVCVAVWRKNDE
NITLETVCHDPKLPYHDFILEDAASPKCIMKEKKK
PGETFFMCSCSSDECNDNIIFSEEYNTSNPD

### Immunoglobulin Fc Domain

The ligand binding domain is linked to a stabilizing protein domain that extends the in vivo plasma half-life of the ligand binding domain. Although in principle any extended length of amino acids may be used as a stabilizing domain, in certain preferred embodiments the stabilizing domain is an immunoglobulin constant (Fc) domain. The Fc advantageously is a human Fc and may be any isotype, although IgG1 and IgG2 isotypes are most commonly used. Suitable Fc domains for this purpose are well known in the art. See, for example, U.S. Pat. No. 5,428,130; Economides et al., Nature Medicine 9:47 (2003); and Czajkowsky et al., EMBO Mol Med. 4:1015-28 (2012). A suitable Fc sequence is shown below. The skilled artisan will recognize that amino acids may be added or deleted from the N- and/or C-termini of the domain, provided that the stabilizing properties of the domain are retained.

(SEQ ID NO: 3)
APELLGGPSVFLFPPKPKDTLMISRTPEVTCVVVD
VSHEDPEVKFNWYVDGVEVHNAKTKPREEQYNSTY
RVVSVLTVLHQDWLNGKEYKCKVSNKALPAPIEKT
ISKAKGQPREPQVYTLPPSRDELTKNQVSLTCLVK
GFYPSDIAVEWESNGQPENNYKTTPPVLDSDGSFF
LYSKLTVDKSRWQQGNVFSCSVMHEALHNHYTQKS
LSLSPGK

### Hinge Region

The ligand binding domain of a naturally occurring immunoglobulin is linked to the Fc domain via a flexible hinge region, and the trap molecules as described herein also contain a modified hinge region fused to the N-terminus of the Fc region. The hinge region can act as a flexible tether, and also contains cysteine moieties that form interchain disulfide bonds. Naturally occurring hinge regions also contain unpaired cysteine residues. It has surprisingly been found that replacement of at least one of these unpaired cysteine residues with a hydrophilic residue (serine, for example) not only provides higher levels of protein expression when the trap molecules are produced in recombinant host cells, but also provides increased activity of the trap in binding TGF-β. Advantageously, at least the first (closest to N-terminus) cysteine of the hinge region is replaced with a hydrophilic residue. This corresponds to the C27 position of a naturally occurring hinge region or position C5 of SEQ ID NO:29 shown below. This modification of the modified hinge is referred to herein as ((C27S)H). An exemplary modified hinge region is shown below, where the underlined serine residue indicates the location of the cysteine to serine substitution:

(SEQ ID NO: 4)
EPKSSDKTHTCPPCP

An exemplary unmodified naturally occurring hinge sequence of human IgG1 is: EPKSCDKTHTCPPCPAPELLGGP (SEQ ID NO:29) (Nezlin, R. General Characteristics of Immunoglobulin Molecules, The Immunoglobulins, 1998)

### Flexible Linker

The trap molecules contain a flexible hydrophilic linker domain that is interposed between the ligand binding domain and the Fc region. Advantageously, the linker also lacks secondary structure and is made up of glycine and serine residues, such as in the well-known (G₄S)ₙ linkers commonly used in, for example, ScFv molecules and the like. The linker may be about 5-35 amino long, and advantageously is about 15-30 amino acids long. An exemplary linker contains 5 repeats of G4S. As described in more detail below, the linker may directly link the C-terminus of the Fc to the N-terminus of the ligand binding domain, or may link the C-terminus of the ligand binding domain to the N-terminus of the hinge region.

### Secretory Signal Sequence

The trap molecules as described herein are produced in recombinant eukaryotic host cells, either in cell culture, or in vivo in host cells that are administered to a patient or subject. Accordingly, the nucleic acid molecules encoding the trap molecules also encode an N-terminal signal peptide that directs the newly synthesized protein into the secretory pathway of the host cell. The signal peptide is cleaved from the rest of the protein during secretion, producing the mature trap protein. Suitable signal peptides are well known in the art. See, for example, von Heijne, Eur. J. Biochem. 133:17-21 (1983); Martoglio and Dobberstein, Trends Cell Biol. 8:410-15 (1988); Hegde and Bernstein., Trends Biochem Sci 31:563-71 (2006). Advantageously, the signal peptide is an immunoglobulin signal peptide. An exemplary signal peptide sequence is shown below:

(SEQ ID NO: 5)
MDWIWRILFLVGAATGAHSAQPA

### Structure of the Trap Molecules

As described above, the hinge region is fused to the N-terminus of the Fc domain, and the ligand binding domain is fused to the hinge-Fc structure via a flexible linker peptide. The ligand binding domain may be positioned either N-terminally or C-terminally relative to the Fc region. When the ligand binding domain is positioned C-terminally, the mature trap protein has the following domain structure:

NH₂-hinge-Fc-linker-TGFβRII-CO₂H.

An exemplary trap molecule with this structure is given as SEQ ID NO:24 herein. In certain embodiments, the trap molecule can have at least 85% identity to SEQ ID NO:24, for example at least 90%, at least 91%, at least 92%, at least 93%, at least 94%, at least 95%, at least 96%, at least 97%, at least 98%, or at least 99% identity to SEQ ID NO:24, with the proviso that a molecule having “X %” identity to SEQ ID NO:24 is always to be understood as having a serine at the position corresponding to position 5 of SEQ ID NO:24, regardless of what other amino acids might be changed relative to the SEQ ID NO:24 sequence.

When the ligand binding domain is positioned N-terminally, the mature trap protein has the domain structure:

NH₂-TGFβRII-linker-hinge-Fc-CO₂H.

An exemplary trap molecule with this structure is given as SEQ ID NO:25 herein. In certain embodiments, the trap molecule can have at least 85% identity to SEQ ID NO:25, for example at least 90%, at least 91%, at least 92%, at least 93%, at least 94%, at least 95%, at least 96%, at least 97%, at least 98%, or at least 99% identity to SEQ ID NO:25, with the proviso that a molecule having “X %” identity to SEQ ID NO:25 is always to be understood as having a serine at the position corresponding to position 166 of SEQ ID NO:25, regardless of what other amino acids might be changed relative to the SEQ ID NO:25 sequence.

### Nucleic Acids and Vectors

Nucleic acid molecules and vectors encoding the trap molecules are provided. Methods of synthesizing the nucleic acid molecules are well known in the art. The nucleic acid sequence may be contained within a vector suited for extrachromosomal replication such as a phage, virus, plasmid, phagemid, cosmid, YAC, or episome. For protein expression the vector is an expression vector, i.e., a vector containing the control elements required for transcription and translation of the inserted protein-coding sequence. Suitable expression systems include mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA, or cosmid DNA. Transcription and translation elements suitable for such host-vector systems are well known in the art. General techniques for preparing nucleic molecules, cloning, and protein expression are described in, for example, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); and “Current Protocols in Immunology” (Coligan, 1991).

In certain embodiments, an inducible promoter containing a TGF-β response element controls trap molecule expression. A suitable vector encoding the trap molecule under the control of the inducible promoter is introduced into a suitable host cell. In certain embodiments, the host cell is chosen on the basis that it can be introduced into a patient or subject that is to be treated with the trap molecule. Expression of TGF-β by a tumor induces production of the trap in the vicinity of the tumor, reducing or eliminating TGF-β activity in that vicinity. This approach advantageously suppresses or inhibits TGF-β activity only in the vicinity of a tumor expressing TGF-β, while avoiding potentially unwanted systemic effects. Suitable TGF-β response elements are known in the art. See, for example, Zhang and Derynck, J. Biol. Chem. 275:16979 (2000); Grotendorst et al., Cell Growth Differ. 7:469-80 (1996); Riccio et al., Mol. Cell Biol. 0.12:1846-55 (1992); see also, SEQ ID NOs:26-28. An expression vector containing a suitable response element is commercially available. See pGL4.28 (Promega, Madison, Wis.) which contains a TGF-β response element as part of a minP promoter element, and which is described in further detail below.

The trap molecule may be produced by introducing a DNA expression vector encoding the trap molecule with N-terminal signal sequence into a host cell, culturing the host cell in media under conditions sufficient to express the trap molecule and allow dimerization of the trap molecule, and purifying the dimeric soluble trap molecule from the host cells or media. When the trap molecule is produced and purified ex vivo the expression vector advantageously contains the DNA encoding the polypeptide under the control of a CMV constitutive promoter.

Alternatively, the host cell may be a mammalian cell, especially a human cell line, which can be introduced into a patient or subject and which produces the trap molecule in situ. In this case, the host cell advantageously contains an expression vector encoding the trap molecule under the control of a promoter that contains a TGF-β response element.

In obtaining variant biologically active TGFβRII, hinge, linker, or Fc domain coding sequences, those of ordinary skill in the art will recognize that the polypeptides may be modified by certain amino acid substitutions, additions, deletions, and post-translational modifications, without loss or reduction of biological activity. In particular, it is well-known that conservative amino acid substitutions, that is, substitution of one amino acid for another amino acid of similar size, charge, polarity, and conformation, are unlikely to significantly alter protein function. The 20 standard amino acids that are the constituents of proteins can be broadly categorized into four groups of conservative amino acids as follows: the nonpolar (hydrophobic) group includes alanine, isoleucine, leucine, methionine, phenylalanine, proline, tryptophan and valine; the polar (uncharged, neutral) group includes asparagine, cysteine, glutamine, glycine, serine, threonine and tyrosine; the positively charged (basic) group contains arginine, histidine and lysine; and the negatively charged (acidic) group contains aspartic acid and glutamic acid. Substitution in a protein of one amino acid for another within the same group is unlikely to have an adverse effect on the biological activity of the protein. In other instance, modifications to amino acid positions can be made to reduce or enhance the biological activity of the protein. Such changes can be introduced randomly or via site-specific mutations based on known or presumed structural or functional properties of targeted residue(s). Following expression of the variant protein, the changes in the biological activity due to the modification can be readily assessed using binding or functional assays.

Sequence identity between nucleotide sequences can be determined by DNA hybridization analysis, wherein the stability of the double-stranded DNA hybrid is dependent on the extent of base pairing that occurs. Conditions of high temperature and/or low salt content reduce the stability of the hybrid, and can be varied to prevent annealing of sequences having less than a selected degree of identity. For instance, for sequences with about 55% G-C content, hybridization, and wash conditions of 40-50 C, 6×SSC (sodium chloride/sodium citrate buffer) and 0.1% SDS (sodium dodecyl sulfate) indicate about 60-70% identity, hybridization, and wash conditions at 50-65° C., 1×SSC and 0.1% SDS indicate about 82-97% identity, and hybridization, and wash conditions of 52° C., 0.1×SSC and 0.1% SDS indicate about 99-100% identity. A wide range of computer programs for comparing nucleotide and amino acid sequences (and measuring the degree of identity) are also available, and a list providing sources of both commercially available and free software is found in Ausubel et al. (1999).

### Protein Expression

For production of purified trap protein, mammalian cells advantageously are used, particularly CHO, J558, NSO, or SP2-O cells. Other suitable hosts include, e.g., insect cells such as Sf9. Non-limiting examples of mammalian cell lines which can be used include CHO dhfr-cells (Urlaub and Chasm, Proc. Natl. Acad. Sci. USA, 77:4216 (1980)), 293 cells (Graham et al., J Gen. Virol., 36:59 (1977)) or myeloma cells like SP2 or NSO (Galfre and Milstein, Meth. Enzymol., 73(B):3 (1981)). Conventional culturing conditions are employed. See, Sambrook, supra. Stable transformed or transfected cell lines can then be selected. Cells expressing a trap molecule can be identified by known procedures. For example, expression of the trap molecule can be determined by an ELISA specific for a domain of the trap molecule, such as the binding domain or the Fc domain and/or by immunoblotting.

For host cells that are introduced into patients and that express the trap molecule in a TGF-β-dependent manner, human host cells are used. A variety of human host cells may be used, including the patient's own cells that can be removed from the patient. Advantageously, however, the host cell is natural killer (NK) cell that is non-MHC restricted and that can therefore be used essentially in any patient without causing an immune response in the patient against the host cell. A suitable NK cell line is the NK-92 cell line, which is known in the art. See, for example U.S. Pat. No. 7,618,817 and Zhang et al., Frontiers in Immunol., vol 8, article 533 (2017). In a particular embodiment, the NK-92 cells are transfected with a suitable nucleic acid construct encoding the trap molecule under the control of a TGF-β inducible control element and then introduced into the patient by, for example, intravenous or intraperitoneal infusion, or direct injection into solid tumors or other cancerous lesions.

Nucleic acid encoding the trap molecule can be introduced into a host cell by standard techniques for transfecting cells. The term “transfecting” or “transfection” is intended to encompass all conventional techniques for introducing nucleic acid into host cells, including calcium phosphate co-precipitation, DEAE-dextran-mediated transfection, lipofection, electroporation, microinjection, viral transduction and/or integration. Suitable methods for transfecting host cells can be found in Sambrook et al., supra, and other laboratory textbooks.

Various promoters (transcriptional initiation regulatory region) may be used to control expression of the molecules described herein. The selection of the appropriate promoter is dependent upon the proposed expression host. Promoters from heterologous sources may be used as long as they are functional in the chosen host. As described above, a promoter incorporating a TGF-β response element or a CMV promoter advantageously are used.

A selective marker is often employed, which may be part of the expression construct or separate from it (e.g., carried by the expression vector), so that the marker may integrate at a site different from the gene of interest. Examples include markers that confer resistance to antibiotics (e.g., bla confers resistance to ampicillin for E. coli host cells, nptII confers kanamycin resistance to a wide variety of prokaryotic and eukaryotic cells) or that permit the host to grow on minimal medium (e.g., HIS4 enables P. pastoris or His- S. cerevisiae to grow in the absence of histidine). The selectable marker has its own transcriptional and translational initiation and termination regulatory regions to allow for independent expression of the marker. If antibiotic resistance is employed as a marker, the concentration of the antibiotic for selection will vary depending upon the antibiotic, generally ranging from 10 to 600 μg of the antibiotic/mL of medium.

The expression construct may be assembled by employing known recombinant DNA techniques (Sambrook et al., 1989; Ausubel et al., 1999). Restriction enzyme digestion and ligation are the basic steps employed to join two fragments of DNA. The ends of the DNA fragment may require modification prior to ligation, and this may be accomplished by filling in overhangs, deleting terminal portions of the fragment(s) with nucleases (e.g., ExoIII), site directed mutagenesis, or by adding new base pairs by PCR. Polylinkers and adaptors may be employed to facilitate joining of selected fragments. The expression construct is typically assembled in stages employing rounds of restriction, ligation, and transformation of E. coli.

Numerous cloning vectors suitable for construction of the expression construct are known in the art (XZAP and pBLUESCRIPT SK-1, Stratagene, La Jolla, Calif., pET, Novagen Inc., Madison, Wis., cited in Ausubel et al., 1999). The selection of cloning vector will be influenced by the gene transfer system selected for introduction of the expression construct into the host cell. At the end of each stage, the resulting construct may be analyzed by restriction, DNA sequence, hybridization, and PCR analyses.

The expression construct may be transformed into the host as the cloning vector construct, either linear or circular, or may be removed from the cloning vector and used as is or introduced onto a delivery vector. The delivery vector facilitates the introduction and maintenance of the expression construct in the selected host cell type. The expression construct is introduced into the host cells by any of a number of known gene transfer systems (e.g., natural competence, chemically mediated transformation, protoplast transformation, electroporation, biolistic transformation, transfection, or conjugation) (see Ausubel or Sambrook, supra). The gene transfer system selected depends upon the host cells and vector systems used.

Standard protein purification techniques can be used to isolate the trap molecule protein of interest from the medium or from the harvested cells. In particular, the purification techniques can be used to express and purify a desired fusion protein on a large-scale (i.e. in at least milligram quantities) from a variety of implementations including roller bottles, spinner flasks, tissue culture plates, bioreactor, or a fermentor.

An expressed trap protein can be isolated and purified by known methods. Typically, the culture medium is centrifuged or filtered and then the supernatant is purified by affinity or immunoaffinity chromatography, e.g. Protein-A or Protein-G affinity chromatography or an immunoaffinity protocol comprising use of antibodies or other binding molecules that bind the expressed trap molecule. The trap molecules can be separated and purified by appropriate combination of known techniques. These methods include, for example, methods utilizing solubility such as salt precipitation and solvent precipitation, methods utilizing the difference in molecular weight such as dialysis, ultra-filtration, gel-filtration, and SDS-polyacrylamide gel electrophoresis, methods utilizing a difference in electrical charge such as ion-exchange column chromatography, methods utilizing specific affinity such as affinity chromatography, methods utilizing a difference in hydrophobicity such as reverse-phase high performance liquid chromatography and methods utilizing a difference in isoelectric point, such as isoelectric focusing electrophoresis, metal affinity columns such as Ni-NTA. See generally Sambrook or Ausubel, supra.

The trap molecule advantageously is substantially pure. That is, the fusion proteins have been isolated from cell substituents that naturally accompany it so that the fusion proteins are present preferably in at least 80% or 90% to 95% homogeneity (w/w). Fusion proteins having at least 98 to 99% homogeneity (w/w) are most preferred for many pharmaceutical, clinical and research applications. Once substantially purified the fusion protein should be substantially free of contaminants for therapeutic applications. Once purified partially or to substantial purity, the soluble fusion proteins can be used therapeutically, or in performing in vitro or in vivo assays as disclosed herein. Substantial purity can be determined by a variety of standard techniques such as chromatography and gel electrophoresis.

### Pharmaceutical Therapeutics

Pharmaceutical compositions are provided that contain trap molecules for use as a therapeutic. The trap molecules can be administered as protein therapeutics or may be provided in situ by secretion from host cells in a TGF-dependent manner.

In one aspect, the trap molecule is administered systemically, for example, formulated in a pharmaceutically-acceptable buffer such as physiological saline. Preferable routes of administration include, for example, instillation into the bladder, subcutaneous, intravenous, intraperitoneal, intramuscular, intratumoral or intradermal injections that provide continuous, sustained, or effective levels of the composition in the patient. Treatment of human patients or other animals is carried out using a therapeutically effective amount of a therapeutic identified herein in a physiologically-acceptable carrier. Suitable carriers and their formulation are described, for example, in Remington's Pharmaceutical Sciences by E. W. Martin.

The amount of the therapeutic agent to be administered varies depending upon the manner of administration, the age and body weight of the patient, and with the clinical symptoms of the neoplasia. For example, the dosage may vary from between about 1 μg trap/kg body weight to about 5000 mg trap/kg body weight; or from about 5 mg/kg body weight to about 4,000 mg/kg body weight or from about 10 mg/kg body weight to about 3,000 mg/kg body weight; or from about 50 mg/kg body weight to about 2000 mg/kg body weight; or from about 100 mg/kg body weight to about 1000 mg/kg body weight; or from about 150 mg/kg body weight to about 500 mg/kg body weight. For example, the dose is about 1, 5, 10, 25, 50, 75, 100, 150, 200, 250, 300, 350, 400, 450, 500, 550, 600, 650, 700, 750, 800, 850, 900, 950, 1,000, 1,050, 1,100, 1,150, 1,200, 1,250, 1,300, 1,350, 1,400, 1,450, 1,500, 1,600, 1,700, 1,800, 1,900, 2,000, 2,500, 3,000, 3,500, 4,000, 4,500, or 5,000 mg/kg body weight. Alternatively, doses are in the range of about 5 mg compound/kg body weight to about 20 mg compound/kg body weight. In another example, the doses are about 8, 10, 12, 14, 16 or 18 mg/kg body weight. Preferably, the trap molecule is administered at 0.5 mg/kg to about 10 mg/kg (e.g., 0.5, 1, 3, 5, 10 mg/kg). In certain embodiments, the trap is administered at a dosage that enhances an immune response of a subject, or that reduces the proliferation, survival, or invasiveness of a neoplastic, infected, or autoimmune cell as determined by a method known to one skilled in the art.

As described above, host cells that secrete the trap molecule in a TGF-β-dependent manner can be administered to patients. The cells can be delivered via intravenous or intraperitoneal infusion, or by other methods known in the art, for example by direct injection into solid tumors or other lesions. In certain embodiments, at least 10³ cells will be administered to the patient, for example at least 10⁴, at least 10⁵, at least 10⁶, at least 10′, at least 10⁸, or at least 10⁹ cells.

### Formulation of Pharmaceutical Compositions

The administration of the trap molecule may be by any suitable method that results in a concentration of the therapeutic that, combined with other components, is effective in inhibiting TGF-β activity. The trap molecule may be contained in any appropriate amount in any suitable carrier substance, and is generally present in an amount of 1-95% by weight of the total weight of the composition (e.g., at least 10% w/w, at least 15% w/w, at least 20% w/w, at least 25% w/w, at least 30% w/w. at least 40% w/w, at least 50% w/w, at least 60% w/w, at least 70% w/w. at least 75% w/w, at least 80% w/w, at least 85% w/w, at least 90% w/w, and at or about 95% w/w). The composition may be provided in a dosage form that is suitable for parenteral (e.g., subcutaneous, intravenous, intramuscular, intravesicular, intratumoral or intraperitoneal) administration route. For example, the pharmaceutical compositions are formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts are initially determined by extrapolating from the amount of compound used in mice or non-human primates, as a skilled artisan recognizes it is routine in the art to modify the dosage for humans compared to animal models. For example, the dosage may vary from between about 1 μg compound/kg body weight to about 5000 mg compound/kg body weight; or from about 5 mg/kg body weight to about 4,000 mg/kg body weight or from about 10 mg/kg body weight to about 3,000 mg/kg body weight; or from about 50 mg/kg body weight to about 2000 mg/kg body weight; or from about 100 mg/kg body weight to about 1000 mg/kg body weight; or from about 150 mg/kg body weight to about 500 mg/kg body weight. For example, the dose is about 1, 5, 10, 25, 50, 75, 100, 150, 200, 250, 300, 350, 400, 450, 500, 550, 600, 650, 700, 750, 800, 850, 900, 950, 1,000, 1,050, 1,100, 1,150, 1,200, 1,250, 1,300, 1,350, 1,400, 1,450, 1,500, 1,600, 1,700, 1,800, 1,900, 2,000, 2,500, 3,000, 3,500, 4,000, 4,500, or 5,000 mg/kg body weight. Alternatively, doses are in the range of about 5 mg compound/Kg body weight to about 20 mg compound/kg body weight. In another example, the doses are about 8, 10, 12, 14, 16 or 18 mg/kg body weight. Preferably, the trap molecule is administered at 0.5 mg/kg-about 10 mg/kg (e.g., 0.5, 1, 3, 5, 10 mg/kg). Of course, this dosage amount may be adjusted upward or downward, as is routinely done in such treatment protocols, depending on the results of the initial clinical trials and the needs of a particular patient.

In certain embodiments, trap molecules as described herein can be formulated with appropriate excipients into a pharmaceutical composition that, upon administration, releases the trap molecule in a controlled manner. Examples include single or multiple unit tablet or capsule compositions, oil solutions, suspensions, emulsions, microcapsules, microspheres, molecular complexes, nanoparticles, patches, and liposomes. Preferably, the trap molecule is formulated in an excipient suitable for parenteral administration.

### Parenteral Compositions

The pharmaceutical composition comprising a trap molecule may be administered parenterally by injection, infusion, or implantation (subcutaneous, intravenous, intramuscular, intratumoral, intravesicular, intraperitoneal) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra. Compositions comprising a trap molecule for parenteral use are provided in unit dosage forms (e.g., in single-dose ampoules). Alternatively, the composition is provided in vials containing several doses and in which a suitable preservative may be added. The composition is in the form of a solution, a suspension, an emulsion, an infusion device, or a delivery device for implantation, or it is presented as a dry powder to be reconstituted with water or another suitable vehicle before use. The composition may include suitable parenterally acceptable carriers and/or excipients and may include suspending, solubilizing, stabilizing, pH-adjusting agents, tonicity adjusting agents, and/or dispersing, agents.

As indicated above, the pharmaceutical compositions containing a trap molecule may be in a form suitable for sterile injection. To prepare such a composition, the trap molecule is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl, or n-propyl p-hydroxybenzoate). In cases where one of the compounds is only sparingly or slightly soluble in water, a dissolution enhancing or solubilizing agent can be added, or the solvent may include 10-60% w/w of propylene glycol.

The present disclosure provides methods of inhibiting TGF-β activity or of treating neoplasia, infectious or autoimmune diseases or symptoms thereof which include administering a therapeutically effective amount of a pharmaceutical composition comprising a trap molecule as described herein to a subject (e.g., a mammal such as a human). Thus, one embodiment is a method of treating a subject suffering from or susceptible to a neoplasia, infectious or autoimmune disease or symptom thereof. The method includes the step of administering to the mammal a therapeutic amount of an amount of a trap molecule sufficient to treat the disease or disorder or symptom thereof, under conditions such that the disease or disorder is treated. Methods of inhibiting TGF-β activity also are provided, by administering to the mammal an amount of a trap molecule sufficient to inhibit TGF-β activity in a patient or subject to a degree that provides a desired therapeutic benefit, such as slowing or inhibiting tumor growth. Identifying a subject in need of such treatment can be in the judgment of a subject or a health care professional and can be subjective (e.g. opinion) or objective (e.g. measurable by a test or diagnostic method).

The therapeutic methods and prophylactic methods in general comprise administration of a therapeutically effective amount of a trap molecule to a subject (e.g., animal, human) in need thereof, including a mammal, particularly a human. Such treatment will be suitably administered to subjects, particularly humans, in which it is desirable to inhibit TGF-β activity. Such patients may suffer from, have, or be susceptible to, or at risk for a neoplasia, infectious disease, autoimmune disease, disorder, or symptom thereof. Determination of those subjects “at risk” can be made by any objective or subjective determination by a diagnostic test or opinion of a subject or health care provider (e.g., genetic test, enzyme or protein marker, biomarker, family history, and the like). The trap molecules may be used in the treatment of any other disorders in which a decrease in TGF-β activity is desired.

Methods of monitoring treatment progress also are provided. The methods include, for example, determining a level of a diagnostic marker or diagnostic measurement (e.g. TGF-β levels) in a subject where the subject has been administered a therapeutic amount of a trap molecule or host cell secreting a trap molecule.

The level of diagnostic marker or measurement determined in the method can be compared to known levels of the marker in either healthy normal controls or in other afflicted patients to establish the subject's disease status. In some cases, a second level of marker in the subject is determined at a time point later than the determination of the first level, and the two levels are compared to monitor the course of disease or the efficacy of the therapy. In certain aspects, a pre-treatment level of marker in the subject is determined prior to beginning treatment according to the methods disclosed herein; this pre-treatment level of marker can then be compared to the level of marker in the subject after the treatment commences, to determine the efficacy of the treatment.

### Combination Therapies

Optionally, the trap molecule is administered in combination with any other standard therapy; such methods are known to the skilled artisan and described in Remington's Pharmaceutical Sciences by E. W. Martin. If desired, the trap molecule is administered in combination with any conventional anti-neoplastic therapy or other therapy, including but not limited to, immunotherapy, therapeutic antibodies, targeted therapy, surgery, radiation therapy, or chemotherapy.

### Kits or Pharmaceutical Systems

Pharmaceutical compositions comprising the trap molecule or host cells expressing the trap molecule may be assembled into kits or pharmaceutical systems for use in inhibiting TGF-β in a subject or patient and thereby ameliorating a disease such as a neoplasia, infectious or autoimmune disease. Kits or pharmaceutical systems may include a carrier, such as a box, carton, tube, having in close confinement therein one or more containers, such as vials, tubes, ampoules, bottles, and the like. The kits or pharmaceutical systems may also comprise associated instructions for using the trap molecule.

### Definitions

“Ameliorate” means to decrease, suppress, attenuate, diminish, arrest, or stabilize the development or progression of a disease.

An “analog” is a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

“Binding to” a molecule means having a physicochemical affinity for that molecule.

“Detect” refers to identifying the presence, absence, or amount of an analyte.

A “disease” means any condition or disorder that damages or interferes with the normal function of a cell, tissue, or organ. Examples of diseases include neoplasia, autoimmune reaction, and viral infection.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease relative to an untreated patient. The effective amount of active compound(s) used to practice the methods disclosed herein for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The terms “isolated”, “purified”, or “biologically pure” refer to material that is free to varying degrees from components which normally accompany it as found in its native state. “Isolate” denotes a degree of separation from original source or surroundings. “Purify” denotes a degree of separation that is higher than isolation.

A “purified” or “biologically pure” protein is sufficiently free of other materials such that any impurities do not materially affect the biological properties of the protein or cause other adverse consequences. That is, a nucleic acid or peptide as described herein is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the nucleotides which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present disclosure further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

By an “isolated polypeptide” is meant a polypeptide that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide. An isolated polypeptide may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide or other molecule having an alteration in expression level or activity that is associated with a disease or disorder.

By “neoplasia” is meant a disease or disorder characterized by excess proliferation. Illustrative neoplasms for which the trap molecules disclosed herein can be used include, but are not limited to leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). In particular embodiments, the neoplasia is multiple myeloma, beta-cell lymphoma, urothelial/bladder carcinoma, or melanoma. As used herein, “obtaining” as in “obtaining an agent” includes synthesizing, purchasing, or otherwise acquiring the agent.

“Reduces” means a diminution of at least 5%, for example at least 10%, at least 25%, at least 50%, at least 75%, or even 100%.

A “reference” means a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

“Specifically binds” means a compound or antibody that recognizes and binds a polypeptide, but which does not substantially recognize and bind other molecules in a sample, for example, a biological sample, which naturally includes a polypeptide.

Nucleic acid molecules useful in the methods disclosed herein include any nucleic acid molecule that encodes a polypeptide or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods disclosed herein include any nucleic acid molecule that encodes a polypeptide or a fragment thereof. Such nucleic acid molecules need not be 100% identical with an endogenous nucleic acid sequence, but will typically exhibit substantial identity. Polynucleotides having“substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

A polypeptide or nucleic acid molecule is “substantially identical” when it exhibits at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison. The terms, “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window. The degree of amino acid or nucleic acid sequence identity for purposes of the present disclosure is determined using the BLAST algorithm, described in Altschul et al. (199) J Mol. Biol. 215:403-10, which is publicly available through software provided by the National Center for Biotechnology Information (at the web address www.ncbi.nlm.nih.gov). This algorithm identifies high scoring sequence pairs (HSPS) by identifying short words of length W in the query sequence, which either match or satisfy some positive-valued threshold score T when aligned with a word of the same length in a database sequence. T is referred to as the neighborhood word score threshold (Altschul et al., supra.). Initial neighborhood word hits act as seeds for initiating searches to find longer HSPs containing them. The word hits are then extended in both directions along each sequence for as far as the cumulative alignment score can be increased. Cumulative scores are calculated for nucleotides sequences using the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below due to the accumulation of one or more negative-scoring residue alignments; or the end of either sequence is reached. For determining the percent identity of an amino acid sequence or nucleic acid sequence, the default parameters of the BLAST programs can be used. For analysis of amino acid sequences, the BLASTP defaults are: word length (W), 3; expectation (E), 10; and the BLOSUM62 scoring matrix. For analysis of nucleic acid sequences, the BLASTN program defaults are word length (W), 11; expectation (E), 10; M=5; N=−4; and a comparison of both strands. The TBLASTN program (using a protein sequence to query nucleotide sequence databases) uses as defaults a word length (W) of 3, an expectation (E) of 10, and a BLOSUM 62 scoring matrix. (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Nat'l. Acad. Sci. USA 90:5873-87). The smallest sum probability (P(N)), provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01.

The terms “treating” and “treatment” refers to the administration of an agent or formulation to a clinically symptomatic individual afflicted with an adverse condition, disorder, or disease, so as to affect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition, or symptoms associated therewith be completely eliminated.

The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

The following examples are put forth so as to provide those of ordinary skill in the art with a complete disclosure and description of how to make and use the assay, screening, and therapeutic methods. These examples are not intended to limit the scope of the invention as claimed herein.

### EXAMPLES

### Materials and Methods:

TGF-β reporter cell line. A TGF-β responsive stable cell line was created by transfecting HEK-293T cells with pGL4.28 (Promega), an expression plasmid containing luciferase driven by a TGF-β response element, using Lipofectamine according to manufacturer's recommended protocol. The transfected cells were selected using hygromycin for two months.

Transfection. TGF-β trap constructs were transiently transfected with Lipofectamine (Thermo Fisher) using the recommended protocol into the TGF-β reporter 293T cell line, then incubated overnight. The cells were then stimulated with TGF-β1, TGF-β2 or mouse TGF-β1 (Cell Signaling Technology) at concentrations as indicated in the figures for 18 hours. Following stimulation, the response was assayed using the Luciferase Assay System (Promega) according to the recommended protocol.

IgG titer measurement: The TGF-β trap IgG fusion titer was measured using the Protein A biosensor on a ForteBio Octet Red96 instrument. HEK-293T cells were transfected with the TGF-β trap constructs and incubated for 18 hours, then the cell culture supernatants were collected and concentrated. The concentrated supernatants were diluted 10-fold in 1×PBS to a final volume of 200 μl and placed in a 96-well plate. The protein A biosensors were incubated in 1×PBS for 10 minutes prior to the measurement. The assay was performed and read at 25° C., and the IgG concentration of each sample was determined by comparing with the standard curve generated with known concentrations of a purified antibody diluted in PBS.

### Example 1: Inhibition of TGF-β Response by Constitutively Active TGF-β Trap Constructs

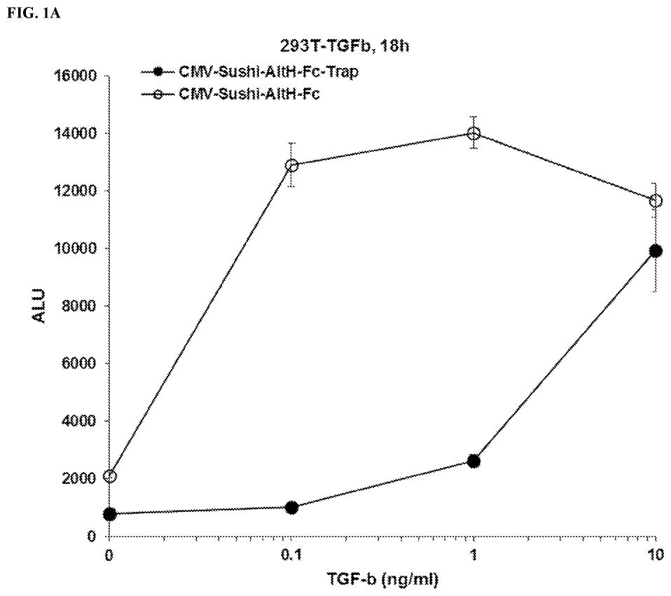

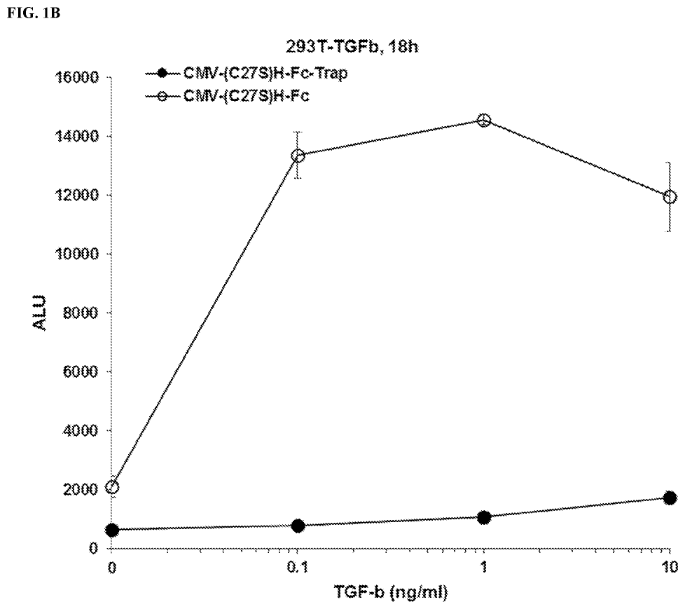

293T cell lines stably expressing TGF-β induced luciferase were transfected with CMV-driven expression constructs comparing the Sushi domain (Sushi), unmodified hinge (AltH), Fc domain (Fc) with or without the TGFBRII (Trap) vs. the modified hinge ((C27S)H), Fc with or without the TGFBRII (Trap) (SEQ ID Nos. 15, 17, 19 and 21; having corresponding DNA sequences of SEQ ID NOs: 14, 16, 18 and 20). Cells were incubated overnight, washed and stimulated with TGF-β at the indicated concentrations. The resulting luciferase activity was measured after 18 hours. The data shown in FIGS. 1A and 1B demonstrate that the trap constructs inhibited TGF-β at low levels (0-1 ng/ml), but only the construct with the modified hinge was effective at high concentrations.

### Example 2: Inhibition of TGF-β Response by TGF-β Inducible Trap Constructs

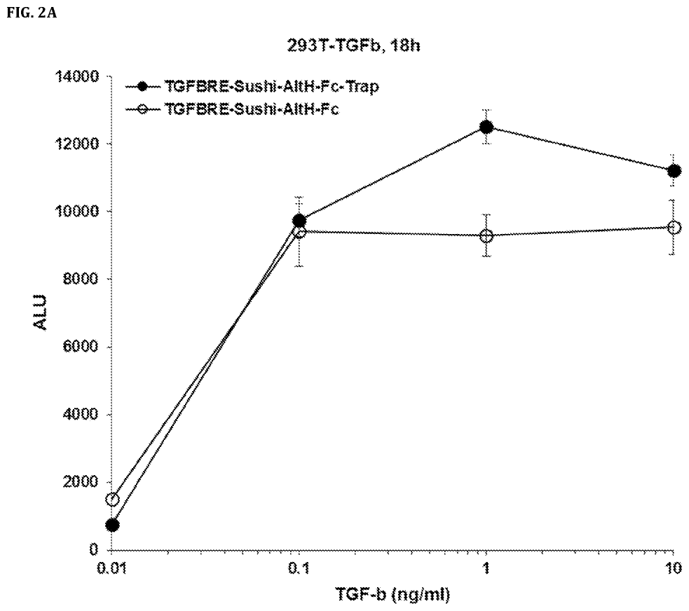

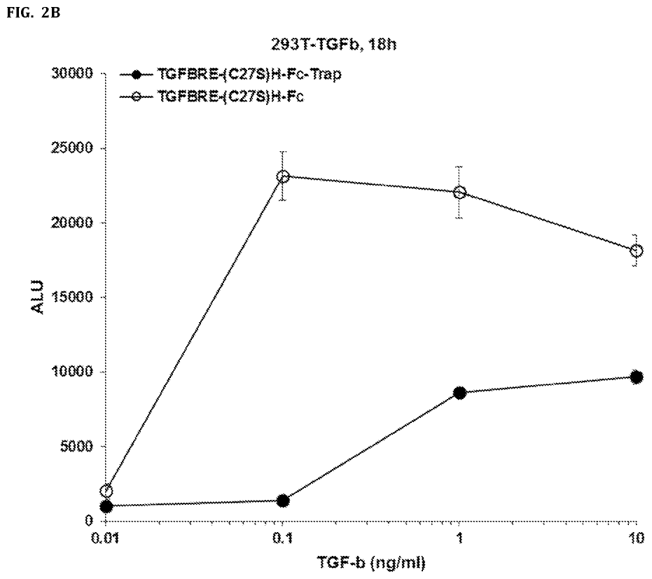

293T cell lines stably expressing TGF-β induced luciferase were transfected with TGF-β response element (TGFBRE)-driven expression constructs comparing the Sushi domain (Sushi), unmodified hinge (AltH), Fc domain (Fc) with or without the TGFBRII (Trap) vs. the modified hinge ((C27S)H), Fc with or without the TGFBRII (Trap) (SEQ ID Nos. 7, 9, 11 and 13; having corresponding DNA sequences of SEQ ID NOs: 6, 8, 10, and 12). Cells were incubated overnight, washed and stimulated with TGF-β at the indicated concentrations. The resulting luciferase activity was measured after 18 hours and the data are shown in FIGS. 2A and 2B. The original construct was unable to block TGF-β activity in the inducible format, whereas the modified construct was effective at low concentrations (0.1 ng/ml), and still demonstrated neutralizing activity at mid to high concentrations (1-10 ng/ml).

### Example 3: Testing TGF-β Trap Expression Constructs in 293T-TGF-β Stables

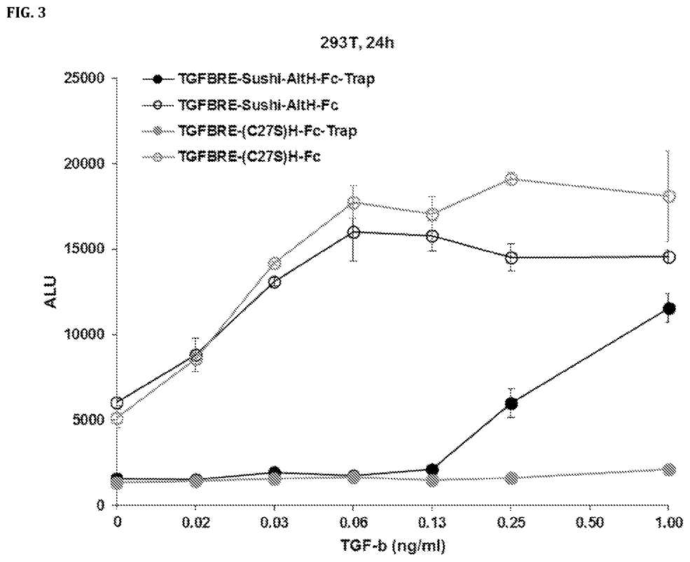

293T cell lines stably expressing TGF-β induced luciferase were transfected with TGF-β response element (TGFBRE)-driven expression constructs with the Sushi domain (Sushi), the unmodified hinge (AltH), FC with or without the TGFBRII (Trap) vs. the modified hinge ((C27S)H), Fc with or without the TGFBRII (Trap) (SEQ ID Nos. 7, 9, 11 and 13; having corresponding DNA sequences of SEQ ID NOs: 6, 8, 10, and 12). Cells were incubated overnight, washed and stimulated with TGF-β at a “low end” titration as indicated. The resulting luciferase activity was measured after 24 hours. The data shown in FIG. 3 indicated that the construct with the modified hinge is significantly more effective.

### Example 4: Testing N-Term Vs C-Term Fusion TGF-β Trap in 293T-TGF-β Stables

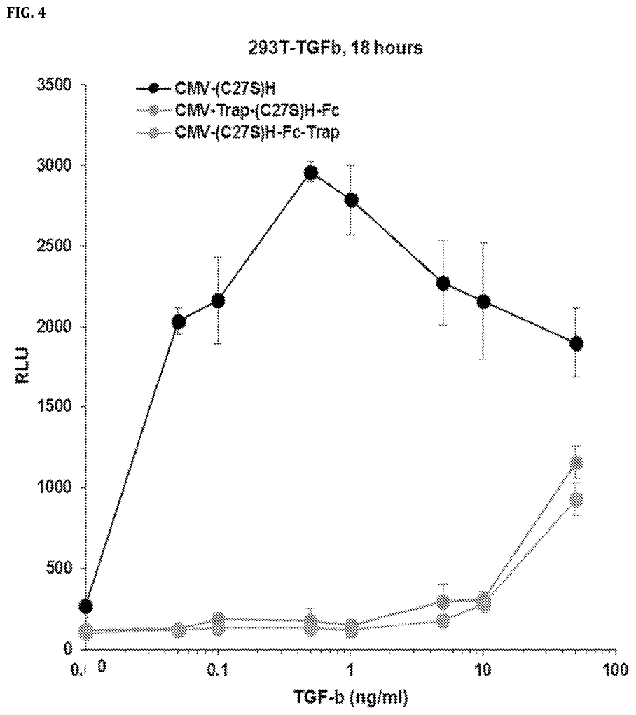

293T cell lines stably expressing TGF-β induced luciferase were transfected with CMV-driven expression constructs comparing the C-terminal (C-term) vs. N-terminal (N-term) TGF-β RII (Trap) Fc-fusion proteins, with the modified hinge ((C27S)H) SEQ ID NOs. 17, 21 and 23; having corresponding DNA sequences of SEQ ID NOs: 16, 20 and 22). Cells were incubated overnight, washed and stimulated with TGF-β as indicated. The resulting luciferase activity was measured after 24 hours. The data in FIG. 4 show no observable difference in activity between the C-terminal vs. N-terminal fusion proteins.

### Example 5: Determining the Ability of the TGF-β Trap can Neutralize TGF-β2

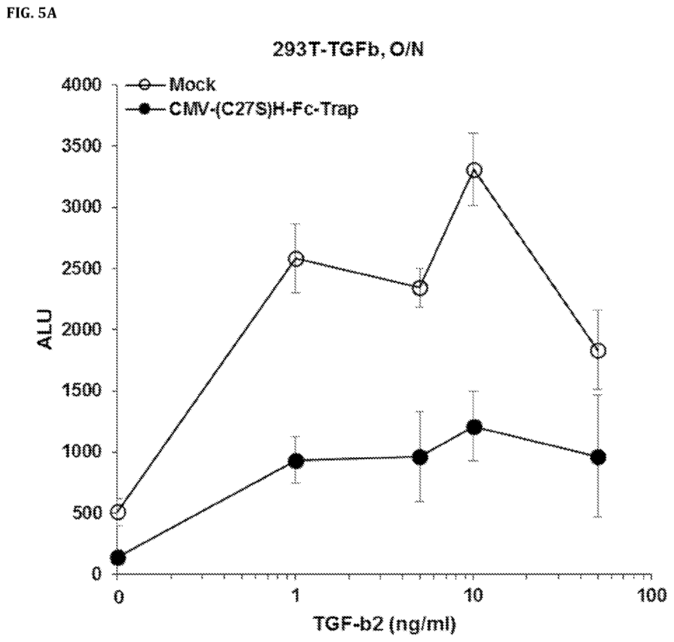

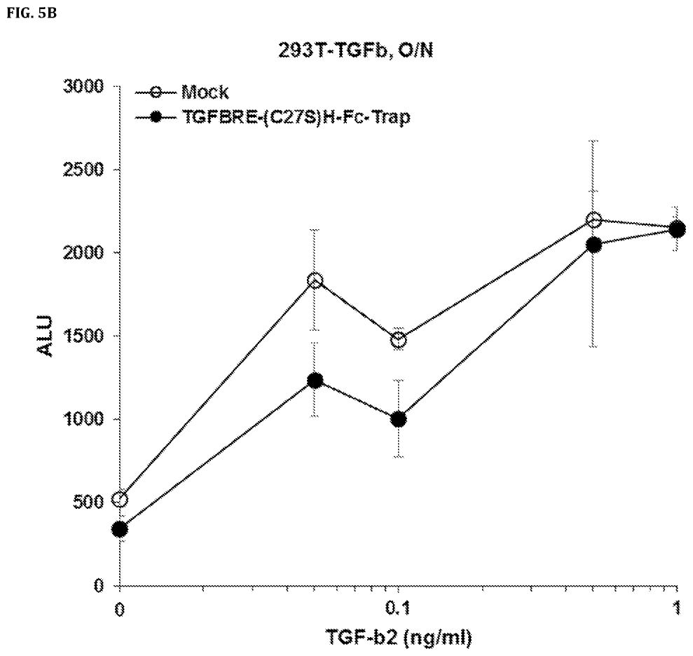

293T cell lines stably expressing TGF-β induced luciferase were transfected with CMV-driven or TGF-β response element (TGFBRE)-driven expression constructs then stimulated with a titration of TGF-β2. Cells were incubated overnight, and the resulting luciferase activity was measured after 24 hours. The data in FIGS. 5A and 5B show a partial ability of the CMV-driven construct and a minimal ability of the TGFBRE-driven construct to inhibit TGF-β2; however, the TGFBRE-driven construct had no discernable ability to neutralize TGF-β2.

### Example 6: Determining the Ability of the TGF-β Trap can Neutralize Mouse TGF-β2

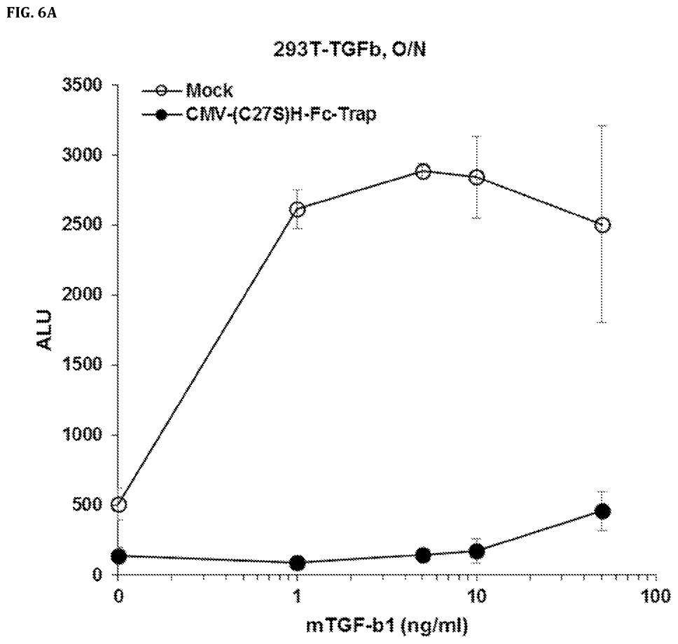

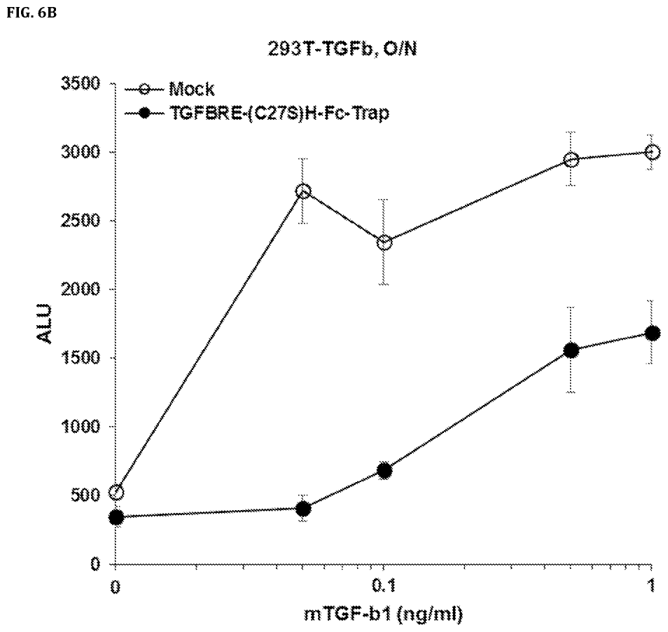

293T cell lines stably expressing TGF-β induced luciferase were transfected with CMV-driven or TGF-β response element (TGFBRE)-driven expression constructs (DNA sequences shown in SEQ ID NOs 8 and 16) then stimulated with a titration of mouse TGF-β1 (mTGF-β1). Cells were incubated overnight, and the resulting luciferase activity was measured after 24 hours. The data shown in FIGS. 6A and 6B indicate that both CMV-driven and TGFBRE-driven trap expression constructs were able to inhibit mouse TGF-β induced luciferase activity.

### Example 7: Determining the Effects of Sushi Domain and Hinge Modification on Production of TGF-β-Trap

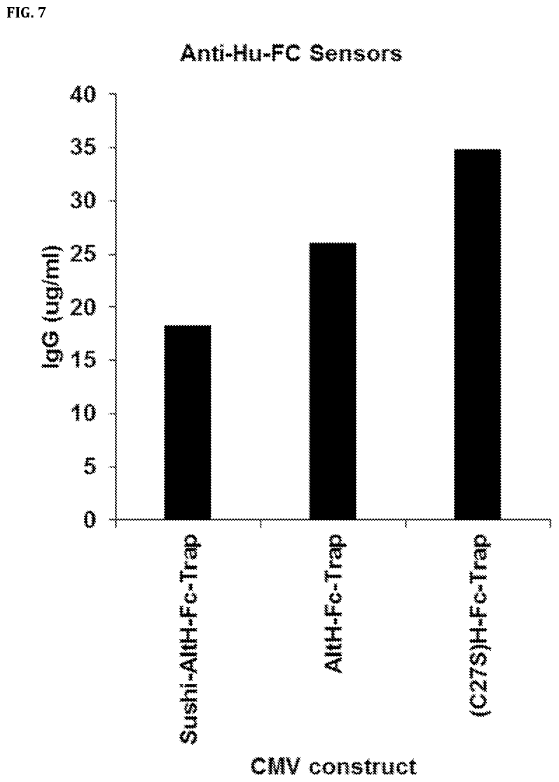

293T cell lines were transfected with CMV-driven TGFBRII trap Fc fusion protein expression constructs containing the Sushi domain and original Unmodified hinge sequence (AltH), no Sushi domain with the AltH, or no Sushi with the modified hinge ((C27S)H). The cells were rested and cultured in serum free medium for 24 hours, and the resulting supernatants were concentrated and the levels of human IgG Fc were measured. The data shown in FIG. 7 show that the no Sushi, C27S hinge product demonstrated the highest concentration.

### OTHER EMBODIMENTS

While this invention has been particularly shown and described with references to particular embodiments thereof, it will be understood by those skilled in the art that various changes in form and details may be made without departing from the scope encompassed by the appended claims.

1.-24. (canceled)

25. TGF-β trap comprising an immunoglobulin constant (Fc) domain fused to a ligand-binding domain of transforming growth factor-beta receptor type 2 (TGFβRII),
wherein said trap does not contain a Sushi domain,
wherein said immunoglobulin Fc domain further comprises an N-terminal immunoglobulin hinge region wherein at least one unpaired cysteine residue of said hinge region is replaced by a serine residue, and wherein said trap has the structure NH₂-hinge-Fc-linker-TGFβRII-COOH.

26. The trap according to claim 25, wherein said TGFβRII is linked to said Fc region via a flexible peptide linker moiety.

27. The trap according to claim 26, wherein said ligand-binding domain of TGFβRII is linked to the carboxy-terminus of said Fc domain via said flexible peptide linker moiety.

28. The trap according to claim 25, wherein the C27 residue of said hinge region is replaced by a serine residue.

29. The trap according to claim 26, wherein said flexible linker comprises G4S repeats.

30. The trap according to claim 29, wherein said linker comprises five G4S repeats.

31. The trap according to claim 25, wherein said trap comprises an amino acid sequence at least 85% identical to SEQ ID NO:24 or at least 85% identical to SEQ ID NO:25.

32. The trap according to claim 25, wherein a peptide signal sequence is fused to the amino terminus of the trap.

33. A nucleic acid molecule encoding a trap according to claim 25.

34. An expression vector comprising a nucleic acid according to claim 33.

35. The vector according to claim 34, wherein said nucleic acid is operably linked to an inducible promoter.

36. The vector according to claim 35, wherein said inducible promoter comprises a TGF-β-inducible promoter.

37. The vector according to claim 34, wherein said nucleic acid is operably linked to a promoter that is constitutively active.

38. The vector according to claim 37, wherein said constitutively active promoter is a CMV promoter.

39. A host cell comprising a vector according to claim 34, wherein said host cell is an IL-2 dependent natural killer cell.

40. method of inhibiting the activity of TGF-β in a subject, the method comprising administering an effective amount of a trap according to claim 25 to a subject in need thereof.

41. A method of treating a neoplasia in a subject, the method comprising administering to a subject in need thereof a therapeutically effective amount of a composition comprising host cells according to claim 39.

42. The method of claim 41, wherein said host cells are administered parenterally, intravenously, peritumorally, or by infusion.

43. The method of claim 40, further comprising administering to the subject an additional therapeutic agent.


SEQUENCE LISTING (29 sequences)

SEQ ID NO: 1 - protein, 566 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 2 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 3 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 15 aa, Artificial Sequence
  Mutant variant of H. sapiens IgG Fc hinge region
EPKSSDKTHTCPPCP

SEQ ID NO: 5 - protein, 23 aa, Homo sapiens
MDWIWRILFLVGAATGAHSAQPA

SEQ ID NO: 6 - DNA, 1557 bp, Artificial Sequence
  TGF-beta RE minP - Sushi - unmodified Hinge - IgG1 Fc - TGFBRII
agtatgtctagactgaagtatgtctagactgaagtatgtctagactgaagcttagacactagagggtatataatggaagctcgacttccagcttggcaatccggtactgttggtaaagccaccatggactggatctggcggattctgtttctcgtgggagctgccacaggcgctcattctgctcagcctgccatcacgtgtcctcctcctatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagagccgaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtaaaggaggaggtggctccggaggcggtggctccggtggaggtggctccggaggtggcggttccggtatccccccccacgtgcagaagtccgttaacaacgacatgatcgtgaccgacaacaacggcgccgtgaagttcccccagctgtgcaagttctgcgacgtgaggttctccacctgcgacaaccagaagtcctgcatgtccaactgctccatcacctccatctgcgagaagcctcaggaggtgtgcgtggctgtgtggcggaagaacgacgagaacatcaccctggagaccgtgtgccacgaccccaagctgccctaccacgacttcatcctggaggacgccgcctcccccaagtgcatcatgaaggagaagaagaagcccggcgagaccttctttatgtgctcctgctccagcgacgagtgcaacgacaacatcatcttctccgaggagtacaacacctccaaccccgactga

SEQ ID NO: 7 - protein, 494 aa, Artificial Sequence
  TGF-beta RE minP - Sushi - unmodified Hinge - IgG1 Fc - TGFBRII
MEARLPAWQSGTVGKATMDWIWRILFLVGAATGAHSAQPAITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 8 - DNA, 1362 bp, Artificial Sequence
  TGF-beta RE minP - C27S Hinge - IgG1 Fc - TGFBRII
agtatgtctagactgaagtatgtctagactgaagtatgtctagactgaagcttagacactagagggtatataatggaagctcgacttccagcttggcaatccggtactgttggtaaagccaccatggactggatctggcggattctgtttctcgtgggagctgccacaggcgctcattctgctcagcctgccgagccgaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtaaaggaggaggtggctccggaggcggtggctccggtggaggtggctccggaggtggcggttccggtatccccccccacgtgcagaagtccgttaacaacgacatgatcgtgaccgacaacaacggcgccgtgaagttcccccagctgtgcaagttctgcgacgtgaggttctccacctgcgacaaccagaagtcctgcatgtccaactgctccatcacctccatctgcgagaagcctcaggaggtgtgcgtggctgtgtggcggaagaacgacgagaacatcaccctggagaccgtgtgccacgaccccaagctgccctaccacgacttcatcctggaggacgccgcctcccccaagtgcatcatgaaggagaagaagaagcccggcgagaccttctttatgtgctcctgctccagcgacgagtgcaacgacaacatcatcttctccgaggagtacaacacctccaaccccgactga

SEQ ID NO: 9 - protein, 429 aa, Artificial Sequence
  TGF-beta RE minP - C27S Hinge - IgG1 Fc - TGFBRII
  MISC_FEATURE (41)..(55): Hinge region
  MISC_FEATURE (56)..(272): Fc region
  MISC_FEATURE (294)..(429): TGFBRII binding domain
MEARLPAWQSGTVGKATMDWIWRILFLVGAATGAHSAQPAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 10 - DNA, 1082 bp, Artificial Sequence
  TGF-beta RE minP - Sushi - Unmodified Hinge - IgG1 Fc
ggtatgtctagactgaagtatgtctagactgaagtatgtctagactgaagcttagacactagagggtatataatggaagctcgacttccagcttggcaatccggtactgttggtaaagccaccatggactggatctggcggattctgtttctcgtgggagctgccacaggcgctcattctgctcagcctgccatcacgtgtcctcctcctatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagagccgaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtaa

SEQ ID NO: 11 - protein, 337 aa, Artificial Sequence
  TGF-beta RE minP - Sushi - Unmodified Hinge - IgG1 Fc
MEARLPAWQSGTVGKATMDWIWRILFLVGAATGAHSAQPAITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 888 bp, Artificial Sequence
  TGF-beta RE minP - C27S Hinge - IgG1 Fc
agtatgtctagactgaagtatgtctagactgaagtatgtctagactgaagcttagacactagagggtatataatggaagctcgacttccagcttggcaatccggtactgttggtaaagccaccatggactggatctggcggattctgtttctcgtgggagctgccacaggcgctcattctgctcagcctgccgagccgaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtaaa

SEQ ID NO: 13 - protein, 272 aa, Artificial Sequence
  TGF-beta RE minP - C27S Hinge - IgG1 Fc
MEARLPAWQSGTVGKATMDWIWRILFLVGAATGAHSAQPAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 2414 bp, Artificial Sequence
  CMV - Sushi - Unmodified Hinge - IgG1 Fc - TGFBRII
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatggactggatctggcggattctgtttctcgtgggagctgccacaggcgctcattctgctcagcctgccatcacgtgtcctcctcctatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagagccgaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtaaaggaggaggtggctccggaggcggtggctccggtggaggtggctccggaggtggcggttccggtatccccccccacgtgcagaagtccgttaacaacgacatgatcgtgaccgacaacaacggcgccgtgaagttcccccagctgtgcaagttctgcgacgtgaggttctccacctgcgacaaccagaagtcctgcatgtccaactgctccatcacctccatctgcgagaagcctcaggaggtgtgcgtggctgtgtggcggaagaacgacgagaacatcaccctggagaccgtgtgccacgaccccaagctgccctaccacgacttcatcctggaggacgccgcctcccccaagtgcatcatgaaggagaagaagaagcccggcgagaccttctttatgtgctcctgctccagcgacgagtgcaacgacaacatcatcttctccgaggagtacaacacctccaaccccgactga

SEQ ID NO: 15 - protein, 477 aa, Artificial Sequence
  CMV - Sushi - Unmodified Hinge - IgG1 Fc - TGFBRII
MDWIWRILFLVGAATGAHSAQPAITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 16 - DNA, 2219 bp, Artificial Sequence
  CMV - C27S Hinge - IgG1 Fc - TGFBRII
acaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatggactggatctggcggattctgtttctcgtgggagctgccacaggcgctcattctgctcagcctgccgagccgaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtaaaggaggaggtggctccggaggcggtggctccggtggaggtggctccggaggtggcggttccggtatccccccccacgtgcagaagtccgttaacaacgacatgatcgtgaccgacaacaacggcgccgtgaagttcccccagctgtgcaagttctgcgacgtgaggttctccacctgcgacaaccagaagtcctgcatgtccaactgctccatcacctccatctgcgagaagcctcaggaggtgtgcgtggctgtgtggcggaagaacgacgagaacatcaccctggagaccgtgtgccacgaccccaagctgccctaccacgacttcatcctggaggacgccgcctcccccaagtgcatcatgaaggagaagaagaagcccggcgagaccttctttatgtgctcctgctccagcgacgagtgcaacgacaacatcatcttctccgaggagtacaacacctccaaccccgactga

SEQ ID NO: 17 - protein, 412 aa, Artificial Sequence
  CMV - C27S Hinge - IgG1 Fc - TGFBRII
MDWIWRILFLVGAATGAHSAQPAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 18 - DNA, 1940 bp, Artificial Sequence
  CMV - Sushi - Unmodified Hinge - IgG1 Fc
gcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatggactggatctggcggattctgtttctcgtgggagctgccacaggcgctcattctgctcagcctgccatcacgtgtcctcctcctatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagagccgaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtaaa

SEQ ID NO: 19 - protein, 320 aa, Artificial Sequence
  CMV - Sushi - Unmodified Hinge - IgG1 Fc
MDWIWRILFLVGAATGAHSAQPAITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 1745 bp, Artificial Sequence
  CMV - C27S Hinge - IgG1 Fc
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatggactggatctggcggattctgtttctcgtgggagctgccacaggcgctcattctgctcagcctgccgagccgaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtaaa

SEQ ID NO: 21 - protein, 255 aa, Artificial Sequence
  CMV - C27S Hinge - IgG1 Fc
MDWIWRILFLVGAATGAHSAQPAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 2228 bp, Artificial Sequence
  CMV - TGFBRII - (G4S)25 Poly Linker - C27S Hinge - IgG1 Fc
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatggactggatctggcggattctgtttctcgtgggagctgccacaggcgctcattctgctcagcctgccatccccccccacgtgcagaagtccgttaacaacgacatgatcgtgaccgacaacaacggcgccgtgaagttcccccagctgtgcaagttctgcgacgtgaggttctccacctgcgacaaccagaagtcctgcatgtccaactgctccatcacctccatctgcgagaagcctcaggaggtgtgcgtggctgtgtggcggaagaacgacgagaacatcaccctggagaccgtgtgccacgaccccaagctgccctaccacgacttcatcctggaggacgccgcctcccccaagtgcatcatgaaggagaagaagaagcccggcgagaccttctttatgtgctcctgctccagcgacgagtgcaacgacaacatcatcttctccgaggagtacaacacctccaaccccgacggaggaggtggctccggaggcggtggctccggtggaggtggctccggaggtggcggttccggtggcggtggctccgagccgaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtaaa

SEQ ID NO: 23 - protein, 416 aa, Artificial Sequence
  CMV - TGFBRII - (G4S)25 Poly Linker - C27S Hinge - IgG1 Fc
MDWIWRILFLVGAATGAHSAQPAIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 389 aa, Artificial Sequence
  Exemplary trap molecule
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 25 - protein, 393 aa, Artificial Sequence
  Exemplary trap molecule
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 38 bp, Mus musculus
ccaccacagccagaccacaggccagacatgacgtggag

SEQ ID NO: 27 - DNA, 50 bp, Homo sapiens
tggagtgtgccagctttttcagacggaggaatgctgagtgtcaaggggtc

SEQ ID NO: 28 - DNA, 67 bp, Homo sapiens
caagtcctagacagacaaaacctagacaatcacgtggctggctgcatgccctgtggctgttgggctg

SEQ ID NO: 29 - protein, 23 aa, Homo sapiens
  MISC_FEATURE (1)..(23): IgG1 Fc hinge region
EPKSCDKTHTCPPCPAPELLGGP